SEQUENCE LISTING (29 sequences)

SEQ ID NO: 1 - DNA, 24 bp, Artificial Sequence
  Synthetic primer
cctacaaggagtaataagacatga

SEQ ID NO: 2 - DNA, 31 bp, Artificial Sequence
  Synthetic primer
gatcttggtttattaacttctgtgatttcat

SEQ ID NO: 3 - DNA, 19 bp, Artificial Sequence
  Synthetic primer
accccaaacagtcgtcaac

SEQ ID NO: 4 - DNA, 26 bp, Artificial Sequence
  Synthetic primer
gaatacagtctaagttgactggtttg

SEQ ID NO: 5 - DNA, 23 bp, Artificial Sequence
  Synthetic primer
gatgaataatcacaagctgctag

SEQ ID NO: 6 - DNA, 24 bp, Artificial Sequence
  Synthetic primer
aacaagttgttcttcttgaagata

SEQ ID NO: 7 - DNA, 22 bp, Artificial Sequence
  Synthetic primer
tgctgaatacagaaaaacagag

SEQ ID NO: 8 - DNA, 20 bp, Artificial Sequence
  Synthetic primer
ctctcctagtctccaccatc

SEQ ID NO: 9 - DNA, 22 bp, Artificial Sequence
  Synthetic primer
tcctagtctccaccactactac

SEQ ID NO: 10 - DNA, 19 bp, Artificial Sequence
  Synthetic primer
gtcaacggttcgactccgt

SEQ ID NO: 11 - DNA, 26 bp, Artificial Sequence
  Synthetic primer
agcagtaatgcagaaaaacagatatg

SEQ ID NO: 12 - DNA, 21 bp, Artificial Sequence
  Synthetic primer
caccatgactttgactggtta

SEQ ID NO: 13 - DNA, 25 bp, Artificial Sequence
  Synthetic primer
gttgcaaacttaattgctactattc

SEQ ID NO: 14 - DNA, 21 bp, Artificial Sequence
  Synthetic primer
agtcgtaacaaggtagccgta

SEQ ID NO: 15 - DNA, 21 bp, Artificial Sequence
  Synthetic primer
tgggttyccccrttcrgaaat

SEQ ID NO: 16 - DNA, 25 bp, Artificial Sequence
  Synthetic oligonucleotide
cgatccaacgcactggccaaaccta

SEQ ID NO: 17 - DNA, 25 bp, Artificial Sequence
  Synthetic oligonucleotide
cggggataccgatctcgggcgcaca

SEQ ID NO: 18 - DNA, 25 bp, Artificial Sequence
  Synthetic oligonucleotide
actacgcaacaccgaacggataccc

SEQ ID NO: 19 - DNA, 25 bp, Artificial Sequence
  Synthetic oligonucleotide
gacattcgcgatcgccgcccgcttt

SEQ ID NO: 20 - DNA, 25 bp, Artificial Sequence
  Synthetic oligonucleotide
ctcggtggtgctgacggtgcaatcc

SEQ ID NO: 21 - DNA, 25 bp, Artificial Sequence
  Synthetic oligonucleotide
ttcggcgctggcgtaaagcttttgg

SEQ ID NO: 22 - DNA, 25 bp, Artificial Sequence
  Synthetic oligonucleotide
ccggctttgaactgctcaccgatct

SEQ ID NO: 23 - DNA, 25 bp, Artificial Sequence
  Synthetic oligonucleotide
tcgtgccggactcgagcaccaatac

SEQ ID NO: 24 - DNA, 25 bp, Artificial Sequence
  Synthetic oligonucleotide
ccccggatagctgacgaggcttacg

SEQ ID NO: 25 - DNA, 25 bp, Artificial Sequence
  Synthetic oligonucleotide
tccggacaggttggggtgcgtttgg

SEQ ID NO: 26 - DNA, 25 bp, Artificial Sequence
  Synthetic oligonucleotide
agcagcagtgacaatgccaccgccg

SEQ ID NO: 27 - DNA, 25 bp, Artificial Sequence
  Synthetic oligonucleotide
agcggtcaccatggccacgaactgc

SEQ ID NO: 28 - DNA, 25 bp, Artificial Sequence
  Synthetic oligonucleotide
gcgatagccagtgccgccaatcgtc

SEQ ID NO: 29 - DNA, 25 bp, Artificial Sequence
  Synthetic oligonucleotide
ccccaaacgtaccaagcccgcgtcg

(19) United States
(12) Patent Application Publication
(10) Pub. No.: US 2009/0253128 A1
(43) Pub. Date: Oct. 8, 2009

(54) NUCLEOTIDE SEQUENCE FOR IDENTIFYING OF ACINETOBACTER BACTERIA, AND METHOD AND KIT OF IDENTIFICATION OF ACINETOBACTER BACTERIA

(75) Inventors: Shu-Ying Li, Taipei City (TW); Yu-Chi Lin, Taipei City (TW)

Correspondence Address: Muncy, Geissler, Olds & Lowe, PLLC, P.O. BOX 1364, FAIRFAX, VA, 22038-1364, US

(73) Assignee: Centers for Disease Control Department of Healthy

(21) Appl. No.: 12/062,201

(22) Filed: Apr. 3, 2008

(51) Int. Cl. C12Q 1/68; C07H 21/02

(52) U.S. Cl. 435/6; 536/24.33

(57) ABSTRACT

Nucleotide primers for identifying Acinetobacter bacteria. The nucleotide primers of the invention are SEQ ID No: 1 to 13, which can be used to effectively and accurately identify various Acinetobacter bacteria. Additionally, the invention further provides a method and kit of identification of Acinetobacter bacteria using the SEQ ID No: 1 to 13.

### BACKGROUND OF THE INVENTION

1. Field of the Invention

The present invention relates to a method for identification of bacteria, and in particular relates to a set of nucleotide primers, method, and kit for identification of Acinetobacter genomic species.

2. Description of the Related Art

Acinetobacter genomic species is classified into at least 32 species (Water Sci. Technol. 46:449-452, 2002), wherein Acinetobacter genomic species 1 (A. calcoaceticus), Acinetobacter genomic species 2 (A. baumannii), Acinetobacter genomic species 3, and Acinetobacter genomic species 13TU are called “A. calcoaceticus-A. baumannii complex, Acb complex”, because they show a highly similar phenotype (J. Clin. Microbiol. 29:277-282, 1991).

In Acb complex, A. baumannii, genomic species 3, and genomic species 13TU are important pathogens encountered in clinical practice (Clin. Microbiol. Infect. 12:826-836, 2006; Appl. Environ. Microbiol. 63:3972-3977, 1997). A. baumannii, genomic species 3, and genomic species 13TU are frequently observed in respiratory therapy apparatus (such as, bottles and coil pipe of respirators, or pulsatile lavage system), and cause urethra infection, bacteremia, meningitis, wound infection, and other nosocomial infections (Clin. Microbiol. Rev. 9:148-165, 1996).

In the past decade, infections caused by multidrug resistant A. baumannii (MDRAB) has increased and affected the use of the drug (Taiwan. Emerg. Infect. Dis. 8:827-832, 2002), and the resistant patterns of MDRAB to various antibiotics is distinct from that of genomic species 3 and genomic species 13TU. Thus, the choice of appropriate therapy has become more and more difficult (J. Antimicrob. Chemother. 59:633-639, 2007; J. Clin. Microbiol. 45:902-905, 2007). To provide a suitable treatment, a rapid and accurate method for identification of Acinetobacter genomic species is necessary.

However, the present commercial identification system (such as Automated Microbic System (Vitek II) and API 20NE bacterial identification system) cannot identify Acb complex. Additionally, although some molecular biology techniques (such as restriction fragment length polymorphism (RFLP) or ribotyping can identify Acb complex, they are time-consuming. Thus, a novel method is required to identify Acinetobacter genomic species.

### BRIEF SUMMARY OF INVENTION

The invention provides a set of nucleotide primers for identifying Acinetobacter genomic species, comprising at least one primer selected from a group consisting of SEQ ID NO: 1, 2, 3, 4, 5, 6, 7, 8, 9, 10, 11, 12, 13, and a complementary sequence thereof, wherein the SEQ ID NO:1 or a complementary sequence thereof identifies A. calcoaceticus, the SEQ ID NO:2 or a complementary sequence thereof identifies A. baumannii, the SEQ ID NO:3 or a complementary sequence thereof identifies Acinetobacter genomic species 3, the SEQ ID NO:4 or a complementary sequence thereof identifies A. haemolyticus, the SEQ ID NO:5 or a complementary sequence thereof identifies A. junii, the SEQ ID NO:6 or a complementary sequence thereof identifies Acinetobacter genomic species 6, the SEQ ID NO:7 or a complementary sequence thereof identifies A. johnsonii, the SEQ ID NO:8 or a complementary sequence thereof identifies A. lwoffii, the SEQ ID NO:9 or a complementary sequence thereof identifies Acinetobacter genomic species 10, the SEQ ID NO:10 or a complementary sequence thereof identifies Acinetobacter genomic species 11, the SEQ ID NO:11 or a complementary sequence thereof identifies A. radioresistens, the SEQ ID NO:12 or a complementary sequence thereof identifies Acinetobacter genomic species 13TU, and the SEQ ID NO:13 or a complementary sequence thereof identifies Acinetobacter genomic species 16.

The invention further provides a method for detecting or identifying Acinetobacter genomic species, comprising: obtaining a biological sample from a subject, wherein the biological sample contains a nucleotide of Acinetobacter genomic species; amplifying a 16S-23S rDNA intergenic spacer of Acinetobacter genomic species using the nucleotide of Acinetobacter genomic species as a template; providing at least one nucleotide primer selected from a group consisting of SEQ ID NO: 1, 2, 3, 4, 5, 6, 7, 8, 9, 10, 11, 12, 13, and complementary sequence thereof; performing a primer extension using the 16S-23S rDNA intergenic spacer as a template and the nucleotide primer as a primer; and determining whether a primer extension product is formed or not, wherein the presence of the primer extension product indicates that the biological sample contains a related Acinetobacter bacterium corresponding to the nucleotide primer.

The invention further provides a kit, comprising: one or more primer(s) selected from a group consisting of SEQ ID NO: 1, 2, 3, 4, 5, 6, 7, 8, 9, 10, 11, 12, 13, and complementary sequence thereof, wherein the primer is hybridized to a 16S-23S rDNA intergenic spacer of Acinetobacter genomic species; and an instruction manual for use of the kit.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows a flow chart showing the steps for identification of Acinetobacter genomic species according to the invention;

FIG. 2 shows a flow chart showing the steps for identification of Acinetobacter genomic species by flow cytometry measurement according to an embodiment of the invention;

FIG. 3 shows that the sensitivity of the method of the invention is 10² cells of Acinetobacter baumannii;

FIG. 4 shows that the method of the invention accurately discriminates AB, 3 and 13TU from the mixture of the three species of A. calcoaceticus-A. baumannii complex, and the sensitivity of the method of the invention is 10 pg; and

FIG. 5 shows that the method of the invention accurately identifies various Acinetobacter genomic species, and no cross reaction is found from non-Acinetobacter species.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides one or more nucleotide primers to accurately detect and/or identify Acinetobacter genomic species, in particular to A. calcoaceticus-A. baumannii complex.

The term “nucleotide” in relation to the present invention includes genomic DNA, cDNA, synthetic DNA, and RNA. Preferably, it means nucleotide or artificial nucleotide of Acinetobacter genomic species. The nucleotide can be a single or double strand nucleotide, or a nucleic acid fragment of complete or partial nucleotides.

The invention provides the nucleotide primers for detecting or identifying Acinetobacter genomic species. The nucleotide primers comprise SEQ ID NO: 1, 2, 3, 4, 5, 6, 7, 8, 9, 10, 11, 12, 13, or the complementary sequence thereof.

The nucleotide primers of the invention are designed based on the 16S-23S rDNA intergenic spacer (ITS) of Acinetobacter genomic species. Accordingly, each nucleotide primer can specifically and complementary bind to a related 16S-23S rDNA ITS of Acinetobacter bacterium.

In the invention, SEQ ID NO: 1 can be used to identify Acinetobacter calcoaceticus; SEQ ID NO: 2 can be used to identify Acinetobacter baumannii; SEQ ID NO: 3 can be used to identify Acinetobacter genomic species 3; SEQ ID NO: 4 can be used to identify Acinetobacter haemolyticus; SEQ ID NO: 5 can be used to identify Acinetobacter junii; SEQ ID NO: 6 can be used to identify Acinetobacter genomic species 6; SEQ ID NO: 7 can be used to identify Acinetobacter johnsonii; SEQ ID NO: 8 can be used to identify Acinetobacter lwoffii; SEQ ID NO: 9 can be used to identify Acinetobacter genomic species 10; SEQ ID NO: 10 can be used to identify Acinetobacter genomic species 11; SEQ ID NO: 11 can be used to identify Acinetobacter radioresistens; SEQ ID NO: 12 can be used to identify Acinetobacter genomic species 13TU; and SEQ ID NO: 13 can be used to identify Acinetobacter genomic species 16.

In one embodiment, the invention provides the nucleotide primers for detecting or identifying A. calcoaceticus-A. baumannii complex. The nucleotide primers select from a group consisting of SEQ ID NO: 1, 2, 3, 12, and a complementary sequence thereof.

In another embodiment, a similar sequence also can be used to identify Acinetobacter genomic species, wherein the similar sequence has at least 60% sequence identity with the nucleotide primer of the invention (SEQ ID NO: 1 to 13), preferably at least 70% sequence identity, even more usually at least 80 to 99% sequence identity.

The nucleotide primer of the invention can be labeled with a ligand or a detectable marker using conventional techniques. Suitable ligand and detectable marker include specific sequence (such as Zipcode, Tag), fluorophores, chromophores, enzyme (such as horseradish peroxidase or alkaline phosphatase), antibody, antigen, biotin, avidin, streptavidin, IgG, protein A, digoxigenin, isotope, and numerous additional receptor-ligand couples known in the art. Other permutations and possibilities will be readily apparent to those of ordinary skill in the art, and are considered as equivalents within the scope of the present invention.

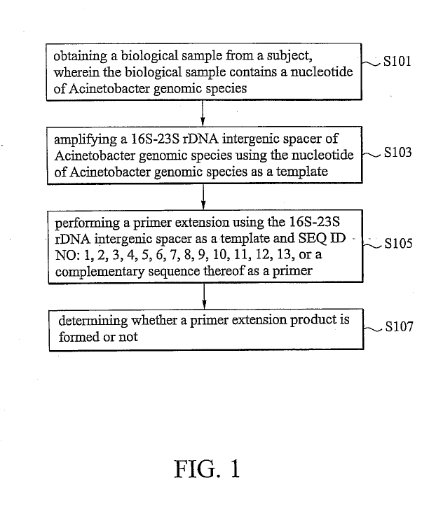

The invention further provides a method for identifying Acinetobacter genomic species. Referring to FIG. 1, in Step 101, a biological sample is collected from a subject, wherein the biological sample contains a target nucleotide. In one embodiment, the target nucleotide is a nucleotide of Acinetobacter genomic species.

The term “subject”, as used herein, means an animal, including a human or non-human mammal, laboratory mammals, livestock and domestic mammals.

A biological sample is any biological material collected from cells, tissues, or organs of the subject. The source of the biological sample may vary depending on the particular symptoms present in the subject to be diagnosed. The biological sample may be analyzed immediately after it is taken, or stored. If stored, the sample may be equilibrated with an appropriate storage buffer, and kept at 4° C., at −20° C., at −70° C., or even in cryogenic liquids, such as liquid nitrogen or liquid helium. In one embodiment, the biological sample may consist of blood, serum, or plasma. In another embodiment, the biological sample may consist of amniotic fluid or milk. In still another embodiment, the biological sample may consist of a biopsy or tissue sample, or a cell suspension. In additional embodiment of the invention, the biological sample may consist of saliva, cerebrospinal fluid, lymph, sweat, mucus, synovial fluid, lacrimal fluid, or other clinical specimens and samples. In still additional embodiment of the invention, the biological sample may be environmental samples including environmental material such as surface matter, soil, water, and industrial samples, as well as samples obtained from food and dairy processing instruments, apparatus, equipment, disposable, and non-disposable items.

The term “target nucleotide” in relation to the present invention includes DNA or RNA/mRNA. For example, the 16S rRNA, 23S rRNA, or intergenic sequence of Acinetobacter genomic species. The target nucleotide can be a single or double strand nucleotide, or a nucleic acid fragment of complete or partial nucleotides.

Referring to Step 3, a 16S-23S rDNA intergenic spacer is amplified by PCR using the target nucleotide as a template and degenerate primers (ITS-F: AGTCG TAACA AGGTA GCCGT A; ITS-R: TGGGT T(C/T)CCC C(A/G)TTC (A/G)GAAA T).

The term “polymer chain reaction, PCR” refers to a DNA polymerase mediated amplification of a given fragment of DNA in a cyclical reaction. In one embodiment, an RNA fragment may be reverse transcribed and amplified by RT-PCR. In the invention, the template, polymerase, dNTP, the concentration of the buffer, and conditions of the PCR (such as temperature or cycle number) are not limited, and can be adjusted based on different samples, apparatus, or primers. One with ordinary skill in the art will select the appropriate protocol to use, and any other relevant experimental parameters. These and many other permutations and possibilities will be readily apparent to those of ordinary skill in the art, and are considered as equivalents within the scope of the present invention.

Referring to Step 105, a primer extension is performed using 16S-23S ITS prepared in Step 103 as a template, and the nucleotide primer of the invention as a primer. The primer extension may be an allele-specific primer extension. In the primer extension process, dATP, dTTP, dCTP, or dGTP is used and labeled with a marker, such as fluorophores, chromophores, enzyme (such as horseradish peroxidase or alkaline phosphatase), antibody, antigen, biotin, avidin, streptavidin, IgG, protein A, digoxigenin, isotope, and numerous additional receptor-ligand couples known in the art.

In one embodiment, the nucleotide primers of the invention (SEQ ID NO: 1 to 13) can be labeled with a specific (characteristic) sequence. For example, Zipcode or Tag.

In another embodiment, the nucleotide primers of the invention (SEQ ID NO: 1 to 13) can be labeled with a marker, such as, beads, His-tag, glutathione, antibody, antigen, biotin, avidin, streptavidin, IgG, protein A, digoxigenin, isotope, and numerous additional receptor-ligand couples known in the art. Additionally, the marker of the dNTP may be distinct from that of the nucleotide primer.

If the nucleotide primer of the invention (SEQ ID NO: 1 to 13) completely complements the 16S-23S ITS fragment, the primer extension is successfully performed to produce a single-strand nucleotide product. However, if the nucleotide primer (SEQ ID NO: 1 to 13) cannot completely complement the 16S-23S ITS fragment, the primer cannot be successfully performed, so that no nucleotide product is produced.

Referring to Step 107, the primer extension product is determined. Because a labeled-dNTP is used in the primer extension process, the primer extension product can be easily detected or determined by any standard method known in the art. If a primer extension product is produced, it indicates that the biological sample contains a related Acinetobacter bacterium corresponding to the nucleotide primer. If no primer extension product is produced, it indicates that the biological sample does no contain the related Acinetobacter bacterium.

In one embodiment, if the dNTP is labeled with a biotin, an avidin or streptavidin can be used to detect the biotin labeled-dNTP. Additionally, the avidin or steptavidin can be labeled with a fluorescent dye, and the amount of fluorescence may be detected by a luminometer, spectrophotometer, or other similar instruments.

In another embodiment, if the dNTP is labeled with an antibody (primary antibody), a secondary antibody can be sued to detect the primary antibody labeled-dNTP. Additionally, the secondary antibody can be labeled with a chromogenic, fluorogenic or chemiluminescent (e.g. horseradish peroxiase or alkaline phosphatase), and the amount of color or fluorescence may be detected by an enzyme-linked immunosorbent assay.

There are many common variations for the detection of the primer extension product. One skilled in the art will select the appropriate protocol to use, depending on the antibody to be detected, the antigen to be used, the source of antigen and/or primary antibody used in the detectopm, and other relevant experimental parameters.

The method of the invention is not interfered with by the DNA of the human blood, so that the method of the invention can be used for a patient's clinical examination and detecting a sample containing mixed Acinetobacter bacteria. Although the concentration of each Acinetobacter bacterium has a large difference, the method of the invention still correctly identifies Acinetobacter bacterium. The sensitivity of the method of the invention is about 10 pg, preferably, 10-100 pg, more preferably, 10 pg to 100 ng.

Furthermore, the method of the invention has a high specificity, because the nucleotide primers of the invention (SEQ ID NO: 1 to 13) specifically determine the related Acinetobacter bacterium, and cannot react with non-Acinetobacter microorganism. For example, Enterobacter (e.g., Enterobacter aerogenes, Enterobacter cloacae, Enterococcus faecalis, Enterococcus faecium), Escherichia coli, Klebsiella pneumoniae, Klebsiella oxytoca, Proteus mirabilis, Pseudomonas aeruginosa, Staphylococcus (e.g., Staphylococcus aureus, Staphylococcus capitis, Staphylococcus epidermidis, Staphylococcus haemolyticus, or Stenotrophomonas maltophilia).

Moreover, the method of the invention provides the advantages of a shorter analysis time than convention methods. The method of the invention not only simultaneously identifies various Acinetobacter species, but the time for analysis of the invention is less than 12 hours, preferably, 10 hours, more preferably, 8 to 9 hours. Thus, the method of the invention is suitably used for the analysis of clinical samples.

The invention further provides a kit for detecting or identifying Acinetobacter genomic species comprising: one or more primer(s) selected from the group consisting of SEQ ID NO: 1, 2, 3, 4, 5, 6, 7, 8, 9, 10, 11, 12, 13, and complementary sequence thereof, wherein the primer is hybridized to a 16S-23S rDNA intergenic spacer of Acinetobacter genomic species; and an instruction manual for use of the kit. Additionally, the kit of the invention further includes all reagents required to determine the Acinetobacter genomic species.

### EXAMPLE

### Example 1

### Identification of the Acinetobacter Genomic Species

Acinetobacter genomic species were bought from the American Type Culture Collection (ATCC), the bacteria names and ATCC accession numbers are shown as in Table 1.

TABLE 1
ATCC accession number of each Acinetobacter genomic species

| Bacteria name | ATCC accession number |
|---|---|
| A. calcoaceticus | ATCC 14987 |
| A. baumannii | ATCC 19606 |
| Genomic species 3 | ATCC 17922 |
| A. haemolyticus | ATCC 17906 |
| A. junii | ATCC 17908 |
| Genomic species 6 | ATCC 17979 |
| A. johnsonii | ATCC 17909 |
| A. lwoffii | ATCC 15309 |
| Genomic species 10 | ATCC 17942 |
| Genomic species 11 | ATCC 11171 |
| A. radioresistens | ATCC 43998 |
| Genomic species 13TU | ATCC 17903 |
| Genomic species 16 | ATCC 17988 |

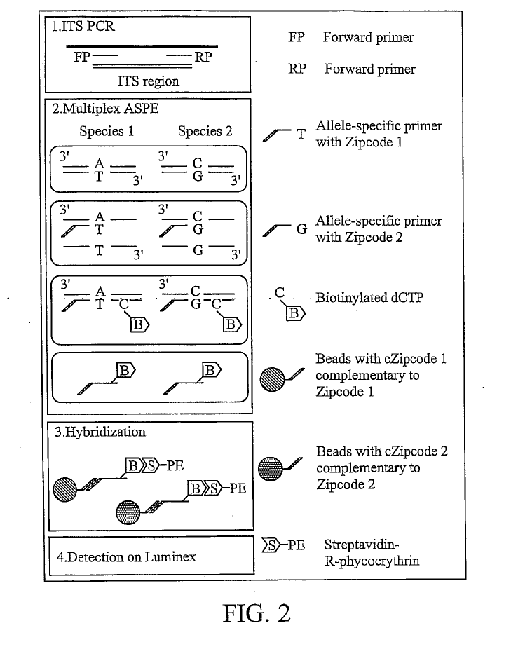

Referring to FIG. 2, the identification of the Acinetobacter genomic species included four procedures. In Procedure 1, 16S-23S ITS fragments of Acinetobacter genomic species were amplified by PCR using the degenerate primers (ITS-F: AGTCG TAACA AGGTA GCCGT A; ITS-R: TGGGT T(C/T)CCC C(A/G)TTC (A/G)GAAA T). Referring to Procedure 2, nucleotide primers (SEQ ID NO: 1 to 13) were labeled with a Zipcode (oligonucleotide of 25 base) (Genome Res. 10:549-557, 2000), respectively. Then, a multiplex allele-specific primer extension (ASPE) (96° C. for 2 min; 96° C. for 30 sec; 55° C. for 1 min; 35 cycles of 72° C. for 2 min; 72° C. for 2 min) was performed to produce an ASPE product using the 16S-23S ITS fragment as a template, the nucleotide primer as a primer, and biotin labeled-dCTP (Invitrogen), wherein the ASPE product was labeled with biotin. Referring to Procedure 3, the beads (Bio-rad) were provided to conjugate the biotin labeled-ASPE product. The each bead was coupled with a specific complementary Zipcode sequence (cZipcode) by a linker, and conjugated with the biotin labeled-ASPE product by the hybridization between the Zipcode and cZipcode. A fluorescent marker (streptavidin-R-phycoerythrin, SAPE) was used to label the biotin labeled-ASPE product. Referring to Procedure 4, color and signal intensity of the SAPE on the biotin labeled-ASPE product were measured with flow cytometry measurement (Bio-Plex 200, Bio-Rad Laboratories, Inc., Hercules, Calif.) according to the manufacturer's instructions. Table 2 shows the primers (nucleotide primers) and means fluorescence intensity (MFI) in Example 1. Table 3 shows the corresponding Zipcode sequences for primers SEQ ID NO: 1-13.

TABLE 2
The primers and mean fluorescence intensity (MFI)

| | Acinetobacter genomic | Mean fluorescence intensity | | |
|---|---|---|---|---|
| Primer | species | Negative | Positive | Min ratio |
| SEQ ID NO: 1 | A. calcoaceticus | 0-26 | 2792-3523 | 107.4 |
| SEQ ID NO: 2 | A. baumannii | 0-445 | 9831-11011 | 22.1 |
| SEQ ID NO: 3 | Genomic species 3 | 7-282 | 669-6256 | 21.5 |
| SEQ ID NO: 4 | A. haemolyticus | 0-31 | 4916-5963 | 158.6 |
| SEQ ID NO: 5 | A. junii | 0-31 | 1445-15698 | 466.0 |
| SEQ ID NO: 6 | Genomic species 6 | 0-45 | 5895-7200 | 131.0 |
| SEQ ID NO: 7 | A. johnsonii | 0-33 | 11210-12082 | 339.7 |
| SEQ ID NO: 8 | A. lwoffii | 57-336 | 3472-3979 | 10.3 |
| SEQ ID NO: 9 | Genomic species 10 | 8-1686 | 7457-7631 | 4.4 |
| SEQ ID NO: 10 | Genomic species 11 | 0-48 | 11084-11632 | 230.9 |
| SEQ ID NO: 11 | A. radioresistens | 0-30 | 5480-9747 | 182.7 |
| SEQ ID NO: 12 | Genomic species 13TU | 0-361 | 12153-13457 | 33.7 |
| SEQ ID NO: 13 | Genomic species 16 | 0-91 | 4962-6162 | 54.5 |
| UniA | All species | NA | 9127-14703 | NA |

Min ratio: the ratio of lowest positive value and the highest negative value.

TABLE 3

The corresponding Zipcode sequences for primers
SEQ ID NO: 1-13

| | | |
|---|---|---|
| Zipcode | Primer | DNA sequences |

| | | |
|---|---|---|
| 20 | SEQ ID NO: 1 | CGATCCAACGCACTGGCCAAACCTA |
| 17 | SEQ ID NO: 2 | CGGGGATACCGATCTCGGGCGCACA |
| 27 | SEQ ID NO: 3 | ACTACGCAACACCGAACGGATACCC |
| 3 | SEQ ID NO: 4 | GACATTCGCGATCGCCGCCCGCTTT |
| 14 | SEQ ID NO: 5 | CTCGGTGGTGCTGACGGTGCAATCC |
| 22 | SEQ ID NO: 6 | TTCGGCGCTGGCGTAAAGCTTTTGG |
| 24 | SEQ ID NO: 7 | CCGGCTTTGAACTGCTCACCGATCT |
| 37 | SEQ ID NO: 8 | TCGTGCCGGACTCGAGCACCAATAC |
| 40 | SEQ ID NO: 9 | CCCCGGATAGCTGACGAGGCTTACG |
| 41 | SEQ ID NO: 10 | TCCGGACAGGTTGGGGTGCGTTTGG |
| 44 | SEQ ID NO: 11 | AGCAGCAGTGACAATGCCACCGCCG |
| 50 | SEQ ID NO: 12 | AGCGGTCACCATGGCCACGAACTGC |
| 49 | SEQ ID NO: 13 | GCGATAGGCAGTGCCGCCAATCGTC |
| 62 | UniA | CCCCAAACGTACCAAGCCCGCGTCG |

### Example 2

### Analysis of Sensitivity (1)

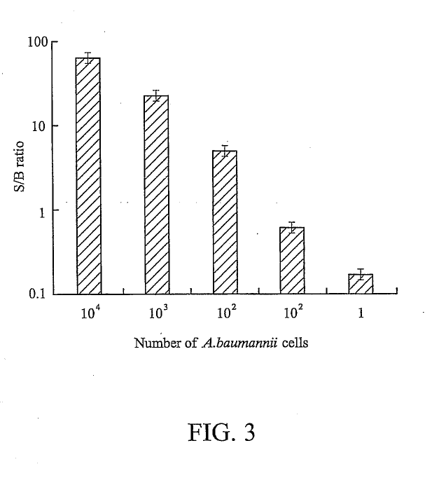

Firstly, 10⁴, 10³, 10², 10, and 1 cells of Acinetobacter baumannii was mixed with serum obtained from a healthy human, and then total DNA was extracted. The total DNA was analyzed by the method as discussed in Example 1, and the absorbance readings were converted to a signal/blank ratio (S/B ratio). An S/B ratio>2 indicates that the method has a high sensitivity for identification of Acinetobacter baumannii. Referring to FIG. 3, the sensitivity of the method for the invention was 10² cells. Accordingly, the method of the invention has a high sensitivity and is not interfered with by DNA of human serum.

### Example 3

### Analysis of Sensitivity (2)

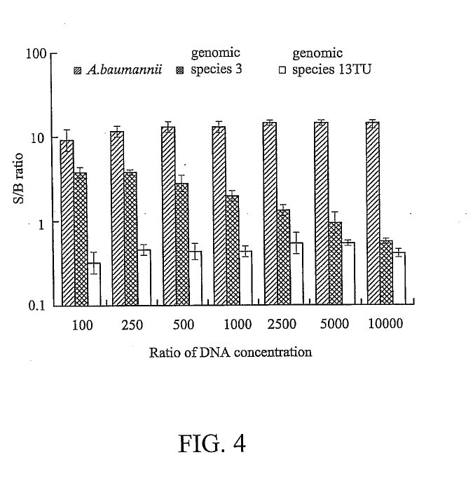

Firstly, Acinetobacter baumannii, Acinetobacter genomic species 3, and Acinetobacter genomic species 13TU were mixed, wherein the DNA concentration of Acinetobacter genomic species 3 and 13TU were constant at 10 pg and did not change, but the DNA concentration of Acinetobacter baumannii was 100 (1 ng), 250 (2.5 ng), 500 (5 ng), 1000 (10 ng), 2500 (25 ng), 5000 (50 ng), and 10000 (100 ng) fold higher than 10 pg, respectively. The signal/blank ratio (S/B ratio) of absorbance was analyzed by the method discussed in Examples 1 and 2. The results of these experiments are shown in FIG. 4, which compare S/B ratio from 1 ng to 100 ng of Acinetobacter baumannii. Referring to FIG. 4, although there was significant difference between the DNA concentrations of each Acinetobacter species in the sample, Acinetobacter baumannii, Acinetobacter genomic species 3, and Acinetobacter genomic species 13TU were still specifically identified by the method of the invention. The data shows that the method of the invention can differentiate a variety of Acinetobacter species in a mixture, and the sensitivity of the method of the invention was 10 pg.

### Example 4

### Analysis of Accuracy

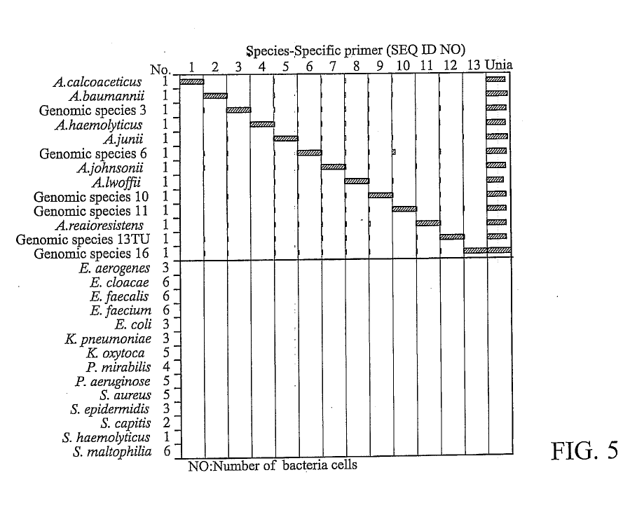

13 Acinetobacter genomic species and 14 non-Acinetobacter species (58 strains) were analyzed by the method discussed in Example 1 above, wherein the non-Acinetobacter included Enterobacter aerogenes, Enterobacter cloacae, Enterococcus faecalis, Enterococcus faecium, Escherichia coli, Klebsiella pneumoniae, Klebsiella oxytoca, Proteus mirabilis, Pseudomonas aeruginosa, Staphylococcus aureus, Staphylococcus capitis, Staphylococcus epidermidis, Staphylococcus haemolyticus, and Stenotrophomonas maltophilia. The 58 non-Acinetobacter stains were isolated from the nosocomial infected patients. The results of these experiments are shown in FIG. 5, filled bar indicated positive reaction, and no bar indicated, no reaction. Referring to FIG. 5, the method of the invention accurately determined various Acinetobacter genomic species and was not interfered with by non-Acinetobacter species.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A nucleotide primer for identifying Acinetobacter genomic species, comprising at least one primer selected from a group consisting of SEQ ID NO: 1, 2, 3, 4, 5, 6, 7, 8, 9, 10, 11, 12, 13, and a complementary sequence thereof, wherein the SEQ ID NO:1 or a complementary sequence thereof identifies A. calcoaceticus, the SEQ ID NO:2 or a complementary sequence thereof identifies A. baumannii, the SEQ ID NO:3 or a complementary sequence thereof identifies Acinetobacter genomic species 3, the SEQ ID NO:4 or a complementary sequence thereof identifies A. haemolyticus, the SEQ ID NO:5 or a complementary sequence thereof identifies A. junii, the SEQ ID NO:6 or a complementary sequence thereof identifies Acinetobacter genomic species 6, the SEQ ID NO:7 or a complementary sequence thereof identifies A. johnsonii, the SEQ ID NO:8 or a complementary sequence thereof identifies A. lwoffii, the SEQ ID NO:9 or a complementary sequence thereof identifies Acinetobacter genomic species 10, the SEQ ID NO:10 or a complementary sequence thereof identifies Acinetobacter genomic species 11, the SEQ ID NO:11 or a complementary sequence thereof identifies A. radioresistens, the SEQ ID NO:12 or a complementary sequence thereof identifies Acinetobacter genomic species 13TU, and the SEQ ID NO:13 or a complementary sequence thereof identifies Acinetobacter genomic species 16.

2. A nucleotide primer for identifying A. calcoaceticus-A. baumannii complex, comprising at least one primer selected from a group consisting of SEQ ID NO: 1, 2, 3, 12, and a complementary sequence thereof.

3. A method for identifying Acinetobacter genomic species, comprising:
obtaining a biological sample from a subject, wherein the biological sample contains a nucleotide of Acinetobacter genomic species;
amplifying a 16S-23S rDNA intergenic spacer of Acinetobacter genomic species using the nucleotide of Acinetobacter genomic species as a template;
providing at least one nucleotide primer selected from a group consisting of SEQ ID NO: 1, 2, 3, 4, 5, 6, 7, 8, 9, 10, 11, 12, 13, and a complementary sequence thereof;
performing a primer extension using the 16S-23S rDNA intergenic spacer as a template and the nucleotide primer as a primer; and
determining whether a primer extension product is formed or not, wherein the presence of the primer extension product indicates that the biological sample contains a related Acinetobacter genomic species corresponding to the nucleotide primer.

4. The method as claimed in claim 3, wherein the subject is a mammal.

5. The method as claimed in claim 3, wherein the subject is a human.

6. The method as claimed in claim 3, wherein the biological sample is selected from serum, blood, plasma, saliva, amniotic fluid, synovial fluid, lacrimal fluid, milk, lymph, urine, and sweat.

7. The method as claimed in claim 3, wherein the nucleotide of Acinetobacter genomic species comprises DNA or RNA.

8. The method as claimed in claim 3, wherein the method of amplifying 16S-23S rDNA intergenic spacer comprises polymerase chain reaction (PCR) or reverse transcription-polymerase chain reaction (RT-PCR).

9. The method as claimed in claim 3, wherein the primer extension is an allele-specific primer extension.

10. The method as claimed in claim 3, wherein the nucleotide primer further comprises a specific sequence.

11. The method as claimed in claim 10, wherein the specific sequence comprises Zipcode or Tag.

12. The method as claimed in claim 3, wherein the primer extension product is labeled with a marker.

13. The method as claimed in claim 12, wherein the marker comprises a fluorescent material, an antibody, an antigen, a biotin, an isotope, a horseradish perosidase, or a digoxigenin.

14. A kit, comprising:
one or more primer(s) selected from a group consisting of SEQ ID NO: 1, 2, 3, 4, 5, 6, 7, 8, 9, 10, 11, 12, 13, and a complementary sequence thereof, wherein the primer is hybridized to a 16S-23S rDNA intergenic spacer of Acinetobacter genomic species; and
an instruction manual for use of the kit.

15. The kit as claimed in claim 14, wherein the primer further comprises a specific sequence.

16. The kit as claimed in claim 14, wherein the specific sequence comprises Zipcode or Tag.
